(19) United States
(12) Patent Application Publication
(10) Pub. No.: US 2014/0017706 A1
(43) Pub. Date: Jan. 16, 2014

(54) FUNCTIONAL ENVIROMICS METHOD FOR CELL CULTURE MEDIA ENGINEERING

(71) Applicants: Rui Manuel Freitas Oliveira, Costa de Caparica (PT); João Miguel Lopes Dias, Lisboa (PT); Ana Raquel Santos Ferreira, Cadaval (PT)

(72) Inventors: Rui Manuel Freitas Oliveira, Costa de Caparica (PT); João Miguel Lopes Dias, Lisboa (PT); Ana Raquel Santos Ferreira, Cadaval (PT)

(73) Assignee: FACULDADE DE CIÊNCIAS E TECNOLOGIA DA UNIVERSIDADE NOVA DE LISBOA, Caparica (PT)

(21) Appl. No.: 13/979,828

(22) PCT Filed: Jan. 13, 2012
(86) PCT No.: PCT/IB2012/050178

§ 371 Date: Oct. 2, 2013

(30) Foreign Application Priority Data
Jan. 14, 2011 (PT) 105484

(51) Int. Cl. C12Q 1/02

(52) U.S. Cl. CPC C12Q 1/025
USPC 435/7.9; 435/29; 506/10; 435/255.5

(57) ABSTRACT

This invention refers to a new method for optimizing the composition of cell culture media. This new method comprises two main stages. In the first stage, a functional enviromics map is built through the joint screening of cell functions and medium factors by the execution of a specific cell culture protocol and exometabolome assays protocol. The functional enviromics map consists of a data array of intensity values of elementary cellular functions against medium factors. In the second stage, optimized cell culture medium formulations are developed that either enhance or repress target elementary cellular functions from columns of the functional enviromics map. The main advantage of this method lies in enabling metabolic engineering through the culture media composition manipulation, wherein an arbitrarily high number of cell functions are optimized through manipulation of medium factors, as opposed to previous methods, which are eminently empirical, are not cell function oriented, and require a much higher number of experiments. Furthermore, this new method is based on cost-effective exometabolome assays and does not require costly intracellular genomic or proteomic assays.

### FIELD OF THE INVENTION

This invention refers to a method for optimising the composition of cell culture media. A method is described for the determination of the optimal values of medium factors, whereby target elementary cellular functions are enhanced or repressed according to user needs. The method comprises the construction of a functional enviromics map through the execution of cell culture experiments and preferably high throughput analytical methods of the exometabolome, followed by medium factors values adjustment on the basis of said function of medium factors.

### BACKGROUND OF THE INVENTION

Cell growth formulations contain hundreds of individual ingredients in water solutions. They generally consist of nutrients, such as peptones, amino acids, meat- and yeast-extracts; minerals and vitamins, inhibitors and solidifying agents. Some of these ingredients may be critical for cell growth or productivity, others may be toxic at certain levels, and many may be involved in complex interactions in the same or competing pathways within the cell. Sera (fetal calf serum, FCS, and fetal bovine serum, FBS), which contain growth factors essential to mammalian cell growth, has been extensively used as medium supplement for animal cell cultures. The use of sera is however being progressively discontinued due to the very stringent constraints imposed by regulatory organizations to the use materials of animal origin for the production of active pharmaceutical ingredients (API). Several documents (e.g. WO2008009642, WO2009149719, US2009061516) disclose serum-free cell culture media formulations which are capable of supporting the in vitro cultivation of animal cells.

Intensive experimentation supported by statistical design-of-experiments (DoE) is the current standard methodology for determining the optimal composition of cell culture media. This methodology is however time-consuming and costly. Media ingredients are screened individually or in small combinations in parallel experiments, which significantly limits the ability to discover complex interactions between many media ingredients. Numerous studies of media optimization using DoE for bacterial, fungal, mammalian and stem cell cultures supported by reactor or shake flasks experiments have been reported ([2], [5], [6], [8]). The most common DoE method is the so called reduced factorial design with two levels of concentrations, which permits a preliminary screening of between five and ten medium factors in a limited number of experiments ([7], [2]). For example, document n^{o} US2008248515 discloses a method that uses 2-level factorial design and the deepest ascent method to determine the optimal composition of a serum-free, eukaryotic cell culture medium supplement. Using DoE, several medium factors are simultaneously compared and their effects are measured and ranked based on measurements of response variables. The response variables are typically the concentrations of metabolites, cells and product concentrations. Once the response variables have been determined, statistical performance parameters, such as analysis of variance (ANOVA), are used to assess the relevance of the measured effects. The medium factors are ranked in relation to their influence, and then the most effective factors are selected and further tested in additional experiments [2]. Finally, a regression model is built to determine optimal levels of the medium factors [2].

The main disadvantage of the statistical DoE method is that, due to its empirical nature, it is cost expansive when applied to many medium factors with potential interactions. To speed-up screening of high numbers of medium factors, costly high-throughput media optimization equipment has been recently developed based on micro-bioreactor technology with the goal of enabling the screening of thousands of nutrient levels or combinations thereof to be run in parallel. Another strategy to decrease the workload and to speed-up medium development is subgrouping the medium ingredients into concentrated compatible formulations as disclosed in document n^{o} NZ243160. Methods that are less empirical and thus faster and cheaper have also been proposed. Document n^{o} MX2009004974 discloses a rational method for cell culture medium design, wherein concentrations of amino acids in the medium are calculated on the basis of protein content of cultured cells, amino acids composition of expressed recombinant proteins and cellular maintenance needs.

There are today more advanced Systems Biology tools that can be applied for less empirical and tendencially mechanistic culture medium design. Kell and co-workers showed that there is a tight link between the exometabolome (the concentration of all extracellular metabolites) and the intracellular state [9]. They showed that exometabolome dynamics provides an informative and accurate “footprint” of cellular metabolic activity and indirectly of genomic and proteomic states. Indeed, the medium transports not only the essential nutrients but also small molecules and proteins involved in gene expression regulation (e.g. [3]). The use of exometabolomics to improve the composition of culture media has however never been described before.

In document n^{o} WO2004101808 a method is described for the development of cell culture medium formulations using genomics and/or proteomics. It describes a method for formulating a cell culture medium comprising detecting a sequence in a cell, the sequence being a nucleic acid sequence (e.g. info about the genome) or an expressed amino acid sequence (e.g. info about the proteome), and formulating a cell culture medium to contain a molecule to modulate the detected sequence or its expression or to modulate a cellular process affected by the detected sequence or its expression, wherein the cell culture medium is formulated without a comparison of the effect of the molecule upon different cell lines or different culture conditions. Such a method presents however three main problems: i) seeking for individual molecular interactions has proved costly and difficult, ii) there is a well-known gap between gene expression, proteome and the cellular phenotype and iii) systematic collection of gene expression or proteome data is difficult and costly. Thus the use of genomics and/or proteomics for systematic medium design may be impracticable in many situations.

An alternative to the modulation of particular biochemical transformations, is function oriented engineering, which is the approach explored in the present document. The metabolism of a cell can be decomposed into elementary cellular functions using elementary flux modes analysis or extreme pathways analysis [35]. An elementary flux mode represents an unique and non-decomposable sub network of metabolic reactions that works coherently in steady state. The complete set of elementary modes represents all operational modes of the cell to realize its function. In particular, the phenotype of a cell, as defined by its fluxome, v, can be expressed as a weighted sum of the contribution of elementary flux modes:

$$v = {λ}_{1} {e}_{1} + {λ}_{2} {e}_{2} + … + {λ}_{K} {e}_{K} = \cdot {\sum }_{i = 1}^{K} {λ}_{i} \times {e}_{i} \qquad ( 1 )$$

with λᵢ the weighting factor of elementary flux mode eᵢ, K the number of elementary flux modes and dim(v)=dim(eᵢ)=q the number of metabolic reactions of the cell. The universe of elementary flux modes is primarily determined by the genome of the cells. Examples of elementary modes determination from genome wide reconstructed metabolic networks can be found in [36]-[38]. The decomposition of the metabolism of cells into elementary flux modes has been previously used to support genetic engineering [39] and functional genomics [40]. An elementary function oriented medium design method, which is the method described in this document, has however never been tempted before. The method described hereinafter is thus called functional enviromics, because it is based on the systematic characterization of the effect of environmental variables (i.e. medium factors) on cellular function. While Functional Genomics is currently a very active field of research in cellular biology, its complementary Functional Enviromics has been referenced in the context of mental health disorders [41] but has never been referenced before in the context cell biology nor has been ever applied for culture media design.

### General Description of the Invention

Previously reported cell culture medium optimization methods are eminently empirical. Because cell culture media comprises many ingredients, a high number of medium factors need to be optimized, eventually requiring the execution of an exponentially growing number of experiments. For example, a two-level factorial design of ten medium factors with potential interactions would require 2̂10=1024 screening experiments. The reason for such a high number of experiments lies in the fact that the biochemical function of medium factors and their interactions are in general not known.

As such, the present invention describes a method for cell culture media development that is primarily focused on the elucidation of the function of medium factors. The general principle adopted in the method of the present invention is schematized in FIG. 1. Whereas the genome of the target cells define its range of elementary flux modes, it is the environment of the cells, i.e. the medium factors, that set the relative intensity of the elementary flux modes to support the phenotype of the cell. Thus the goal of the method is in a first stage to identify how medium factors control the relative intensity of elementary flux modes, i.e. functional enviromics analysis. This is accomplished through the execution of an experimental protocol, wherein an array of cell culture experiments is performed, wherein perturbations to baseline medium factors are introduced and response exometabolomic data is acquired and analyzed. The so acquired data is processed into the form of a functional enviromics map of elementary cellular functions against medium factors. In a second stage, from the functional enviromics map, optimized medium formulations are developed that either enhance or repress target elementary cellular functions in order to enforce a desired phenotype.

Such a method presents several benefits in relation to the currently used methods:
It enables to engineer the metabolism of cells by the adjustment of several target metabolic objectives simultaneously.
It is less empirical and thus can be more easily generalised or extrapolated to different cell species sharing a given group of genes. As the function of genes tends to be conserved among different species also the function of environmental variables tends to be conserved among different species.
The medium design is thus based on knowledge, which can be augmented over time without the need to repeat previously explored conditions.
Once a sufficient knowledge base is built, medium designs require a much smaller number of experiments. In theory, if the knowledge base is sufficiently accurate, a single step design is required to optimise the metabolism of the target cells. As shown in examples 4 and 5, a single design step may result in improvements in the range of 60%-100% in protein productivity.

### DETAILED DESCRIPTION OF THE INVENTION

Hereinafter, the best mode for carrying out the present invention is described in detail.

A distinctive feature of the method of the present invention is that medium factors and elementary cellular functions are joint screened through a particular experimental protocol to extract data, which is then processed into the form of a Functional Enviromics map. Before the experimental protocol is executed, it is mandatory to clearly state the biological structure to which the medium will be designed, hereon referred to as “target biological structure”. It can be a whole cell, an organelle, or a coherent set of biochemical reactions that represent a given cellular function. Said target biological structure is represented by q biochemical reactions and associated genes and proteins in case they are known. Once this structure is clearly stated, then the underlying working set of N medium factors and K elementary cellular functions are established.

The culture medium formulation is defined by the values of N medium factors:

Culture medium formulation={FACⱼ}, j=1, . . . ,N,

with FACⱼ the value of medium factor j. Said medium factors may be physicochemical properties (e.g. temperature, osmolality or ionic strength), concentration or rate of release of known or unknown molecular species, or concentration or rate of release of mixtures with known or unknown composition.

The target biological structure is defined by q biochemical reactions which are transformed into K elementary cellular functions:

Target biological structure={eᵢ}, i=1, . . . ,K.

The transformation of q biochemical reactions into K elementary cellular functions can be obtained by applying public domain bioinformatics algorithms [17], such as elementary flux modes analysis or extreme pathways analysis. Example 1 illustrates how the elementary cellular functions are obtained for the yeast Pichia pastoris X33.

Once this general structure is known, a method comprising four steps is applied as follows (see FIG. 2):

Step 1—Array of Cell Cultures

Step 1.1—Execute at least N+1 (number of medium factors plus one) cell culture experiments with varying culture medium composition in shake flasks, T-flasks or reactors, but preferably in high-throughput cultivation equipment such as micro plates, micro bioreactors or phenotype microarrays. The medium composition screened in each experiment is defined in a way to generate adequate experimental data for the purpose of linear regression of elementary cellular function weighting factors against culture medium factors values. Example 2 illustrates this procedure for the case of 11 medium factors using the D-optimal design method for linear function identification, wherein 24 independent experiments are defined, which consist of two-level combinations of medium factor values. Further, such medium formulations might include labelled substrates, such as ¹³C-substrates for analytical purposes, namely to facilitate the screening of elementary cellular functions, and/or protein inhibitors or activators, or interference ribonucleic acid or other functional molecules that knock up or knock down elementary cellular functions.

Step 1.2—Acquire initial and end-point exometabolome data, i.e. concentrations of a high number of metabolites, and also cellular concentration data, for each cell culture performed using preferably fast and high-throughput analytical techniques such as 1H-NMR, 13C-NMR or other NMR technique, or chromatography coupled to mass spectrometry such as GC-MS or LC-MS or mass spectrometry techniques alone. This analysis can be complemented with more traditional metabolite specific analytical methods such as enzymatic kits or high-performance liquid chromatography (HPLC).

Step 1.3—Pre-screening of active elementary cellular functions by linear regression of elementary cellular functions weighting factors against medium factors. These weighting factors are obtained from the initial and endpoint exometabolome data. First, determine the rate of change of metabolites according to formula (2) or any other approximate method to calculate rate of change of a property from time series measurements of said property,

$$v = \frac{1}{{C}_{X}} \frac{d {C}_{Mi}}{d t} \approx \frac{1}{{C}_{X}} \frac{Δ {C}_{Mi}}{Δ t} , \qquad ( 2 )$$

with C_{Mi} the concentration of metabolite i, C_{X} the cellular concentration and t the culture time.

Then, for all K elementary cellular functions, determine the potential maximum eᵢ weighting factor value by applying the following formula:

λᵢ=veᵢ, i=1, . . . ,K (3)

Then, linearly regress λᵢ against medium factors values according to formula (4)

$${λ}_{i} = \cdot {\sum }_{j = 1}^{N} {I}_{j , i} \times {FAC}_{j} \qquad ( 4 )$$

With I_{j,i} the regression parameters which represent the intensity of activation of cellular function eᵢ by culture medium factor FACⱼ. Then rank elementary cellular functions from low to high correlation coefficient of λᵢ against medium factors values. Alternatively, rank the elementary cellular functions from low to high explained variance of rate data, v. Then select a subset of K′<<K cellular functions with the highest correlation coefficients and highest explained variance for further screening in step 1.4.

Step 1.4—Execute at least K′+1 cell culture experiments with varying culture medium composition in a similar fashion to step 1.1. But the strategy is now to screen combinations of low and high values of elementary cellular function weighting factors. Ideally 2̂K experiments should be performed to screen all possible combinations. In practice, not all the K elementary cellular functions can be screened as their number can easily increase to the range of millions. However, in the previous step 1.3, a pre-selection of the K′ elementary cellular functions with highest contribution to the cellular phenotype and highest correlation with medium composition is obtained, which rarely rises above K′=20. For this second run of experiments, the culture medium compositions are modified around the baseline formulation using the intensity values determined in the previous step. These intensity values define the direction of change of medium factor values in order to up- or down regulate elementary cellular functions according to formula (5):

Δ(λᵢ)=·I_{j,i}×Δ(FACⱼ). (5)

Step 2—Functional Enviromics Map

For the totality of experiments performed in step 1, organize medium composition data in a data matrix X={Fac_{i,j}}, a M×N matrix of M medium formulations, with M≧N+K′+2, and respective measured flux data, R={vᵢ}, a M×q matrix of measured fluxes. Then determine a subset of elementary cellular functions among the whole set of elementary cellular functions which is tightly linked to the medium factors and determine their weighting factor to the observed cellular phenotype, R={vᵢ}, by regression analysis of R={vᵢ} against medium composition data X={Fac_{i,j}} satisfying the following criteria:
a. Maximise the captured variance of exometabolome data and/or rate of change of exometabolome datam R={vᵢ}, and/or other derived information from exometabolome data.
b. Maximise correlation between elementary cellular functions weighting factors and medium factors or transformations of their values.
c. Minimise the number of elementary cellular functions required to capture a given variance of exometabolome data and/or rate of change of exometabolome data R={vᵢ}, and/or other derived information from exometabolome data, i.e. minimize redundancy.

These criteria can be fulfilled by maximizing the covariance between medium composition data, X={Fac_{i,j}}, and respective measured flux data, R={vᵢ}, according to formula:

$${Maximize}_{I} cov ( X , R ) \\ s . t . \{ \begin{matrix}R = Λ \times {EM}^{ T} \\ Λ = X \times {I}^{T}\end{matrix} \qquad ( 6 )$$

with EM={eᵢ} a q×K matrix of K elementary cellular functions, eᵢ (dim(eᵢ)=q), Λ={λᵢ} a M×K matrix of weight vectors λᵢ of elementary cellular functions (dim(λᵢ)=M) and I={I_{i,j}} a K×N matrix of intensity parameters, which are the degrees of freedom to solve formula (6). Several methods can be used to solve formula (6). One efficient method consists in a one by one elementary cellular function decomposition according to formulas (7a-c)

X=T·W^{T}+EF_{X} (7a)

R=Λ×EM^{T}+EF_{R} (7b)

Λ=T·B^{T}+EF_{Λ} (7c)

with EFᵢ residuals matrices that are minimized, W a matrix of loading coefficients and B a matrix of regression coefficients. Finally, the intensity matrix I is given by

I=·B·W^{T} (8)

The result of this procedure is the discrimination of the subset of elementary cellular functions that is tightly linked with medium composition. The information can finally be organized in a N×K data array, called functional enviromics map (FEM):

Functional Enviromics map=I^{T}={I_{j,i}}, j=1, . . . ,N i=1, . . . ,K

The rows represent medium factors, columns represent the universe of elementary cellular functions and I_{j,i} the relative “intensity” of up- or down-regulation of elementary cellular functions i by medium factor j.

Accomplishing these steps require screening a high number of medium formulations, which is costly. However, once done, the fingerprint of the cell in terms of environment-function properties is established. Moreover, functional enviromic maps are conserved in the same way that the function of genes is conserved. As such, the patterns identified for a given function within different genotypes are expected to show critical similarities that reflect the deterministic link between genes and environment. Example 3 illustrates the construction of a functional Enviromics map for the yeast P. pastoris X33.

Step 3—Optimized Culture Media Formulation

In culture media design, ideally one should be able to fine tune cellular functionality according to user needs. Combining the information of desired functionality and that of Functional Enviromics Maps it is possible to deduce aprioristic rules about how to tune medium composition in relation to some baseline formulation in a way to enforce the desired functionality. This allows reducing drastically the number of experiments for culture media design. In limit, if the information in functional Enviromics maps is sufficiently accurate, a single design step results in a quasi optimal culture medium formulation. More specifically the procedure is as follows:

Each column of FEM matrix holds information of how the baseline medium factor values should be adjusted either to enhance or repress a particular elementary cellular function. More specifically, the new medium factor values should be changed according to the following formula:

FACⱼ^{opt}=(1+ηᵢIᵢⱼ)FACⱼ⁽⁰⁾ (9)

with FACⱼ⁽⁰⁾ the baseline value of medium factor j, FACⱼ^{opt} the optimized value of medium factor j, I_{j,i} the intensity value of the jth row and ith column of the functional Enviromics map and ηi the desired enhancement factor of elementary cellular function i (design parameter). Cellular function specific medium supplementation formulations can be deduced from columns of the functional enviromics map. Alternatively, globally optimized medium formulations can be obtained by applying enhancement factors to several elementary cellular functions simultaneously.

Step 4—Final Validation Step

The newly optimized culture media formulation is screened in additional culture experiments as describe in steps 1. The number of experiments is however now much lower, typically triplicates of a given optimized medium formulation.

At the end of step 4 it is expected productivity and/or product quality or overall culture performance gains far beyond the baseline medium formulation. Increase in productivity is obviously case dependent. Examples 4, 5 and 6 show how productivity increases in range of 60%-100% can be obtained for a recombinant P. pastoris X33 strain.

### SUMMARY OF THE INVENTION

The object of the present invention is a method for determining optimal cell culture medium composition comprising the following steps:

a) state the biological structure to which the medium will be designed;

b) establish the working set of N medium factors and K elementary cellular functions of previously stated biological structure;

c) build a Functional Enviromics Map of K elementary cellular functions against N medium factors of target biological structure by
execute a first run of cultivation experiments comprising a number of cultivations equal or higher than the number of medium factors plus one (N+1), wherein each cultivation is performed in a different culture medium composition, wherein combinations of low and high levels of medium factors are screened;
acquire initial and end-point biomass, product and exometabolome data for each cultivation experiment performed;
determine a subset of K′ active elementary cellular functions that show high correlation coefficients with medium factors values by regression analysis of exometabolome data or derived exometabolome data against medium composition data;
execute a second run of cultivation experiments comprising a number of cultivations equal or higher than the number of active cellular functions plus one (K′+1), wherein each cultivation is performed in a different culture medium composition, wherein combinations of low and high intensity levels of elementary cellular functions are screened;
build a functional enviromics map from the data collected in previous steps by maximizing the covariance between medium composition data, X={Fac_{i,j}}, and respective measured flux data, R={vⱼ}, according to formula:

$${Maximize}_{I} cov ( X , R ) \\ s . t . \{ \begin{matrix}R = Λ \times {EM}^{ T} \\ Λ = X \times {I}^{T}\end{matrix} \qquad ( 6 )$$

and organize the data in the form of a functional Enviromics map, wherein intensity values are put into the form of a N×K data array:

Functional Enviromics map=I^{T}={I_{j,i}};

d) optimization of culture media composition using functional enviromics maps by
formulate elementary cellular function specific medium compositions using the functional enviromics data matrix, wherein variations in medium factor values Δ(FACⱼ) result in variations of the relative weight of elementary cellular functions Δ(λᵢ) according to the formula

Δ(λᵢ)=·I_{j,i}×Δ(FACⱼ) (5)
formulate culture medium compositions to enhance or repress a single elementary cellular function i using formula (5) applied to the ith column of the functional enviromics map;
formulate culture medium compositions to engineer cellular metabolism by enhancing or repressing critical sets of elementary cellular function using formula (5) applied to multiple column of the functional enviromics data array simultaneously.

In a preferable embodiment:
the target biological structure can be a cell tissue, a whole cell, an organelle, or a coherent set of metabolic reactions that represent a given cellular function;
the medium factors are physicochemical properties, concentrations and/or rate of release of essential nutrients and/or micronutrients and/or functional molecules.
the physicochemical properties are temperature, pressure, pH, ionic strength and/or osmolality.

In another preferable embodiment the culture medium formulation is determined by the values of N medium factors using the following formula:

Culture medium formulation={FACⱼ}, j=1, . . . ,N,

with FACⱼ the value of medium factor j; and the target biological structure is determined by K elementary cellular functions using the following formula:

Target biological structure={eᵢ}, i=1, . . . ,K.

In another preferable embodiment the elementary cellular functions are obtained from a biochemical network of said target biological structure, wherein the biochemical network is sub-divided into K functional sub-networks comprising a subset of biochemical transformations, wherein such sub-networks are obtained manually and/or automatically by applying elementary flux modes algorithms or extreme pathways algorithms or other null space analysis algorithms or other null space convex analysis algorithms of the metabolic network of said target biological structure.

In another preferable embodiment the elementary cellular functions are obtained from genome scale reconstruction of the biochemical network of said target biological structure, wherein the working set of K elementary cellular functions may be pre-reduced using transcriptome data and/or proteome data and/or endometabolome data and/or thermodynamic data in case these data are available.

In another preferable embodiment the functional enviromics map is determined by serial and/or parallel culture experiments performed in shake-flasks, T-flasks, reactors, microplates, microbioreators or phenotype microarrays. Preferably the functional enviromics map is determined by exometabolome assays, comprising, analysis of the supernatant by NMR technique, such as 1H-NMR or 13C-NMR, by chromatography techniques, such as liquid chromatography (LC) or gas chromatography (GC), mass spectrometry techniques (MS) or chromatography coupled to mass spectrometry, such as GC-MS or LC-MS.

In another preferable embodiment a reduced set of active elementary cellular functions are identified by linear or nonlinear regression analysis, wherein variance or co-variance of exometabolome data or derived exometabolome data is maximized, wherein correlation between exometabolome data or derived exometabolome data and medium factors values is maximised, wherein elementary cellular functions are ranked according to their correlation or sensitivity to medium factors values.

In another preferable embodiment the functional enviromics map is determined by a high-throughput automated system, wherein cultivation devices, analytical exometabolome devices and computational algorithms are interfaced in a physical device to produce high-throughput functional enviromics maps.

With the method of the present invention the product elementary flux mode enhances between 60 to 100%.

Another aspect of the present invention is the use of the method described above for optimization of the composition of cell culture media of Plantae or Animali cell lines or other eukaryotic unicellular or multicellular organism such as Yeasts and Fungi, preferable for optimization of the composition of cell culture media of prokaryotic organism and for optimization of the composition of cell culture media of stem cells.

Another aspect of the present invention are biomarkers identifiers of elementary cellular functions. Medium components, excreted or secreted by the cell and detected in the exometabolome, that are found to be strongly correlated with a single elementary cellular function using the method of the present invention, can serve as biomarkers of that elementary cellular function.

Another aspect of the present invention are drug design systems targeting elementary cellular functions comprising the method described above. A compound that is found to strongly correlate with elementary cellular functions associated to a disease condition can constitute a drug candidate to treat that disease.

### BRIEF DESCRIPTION OF THE FIGURES

FIG. 1 represents the conceptual approach of the method of the present invention, namely for function oriented culture media engineering, in which λᵢ represents elementary function weighting factors, controlled by medium factors, and eᵢ represents elementary cellular functions, imposed by genes.

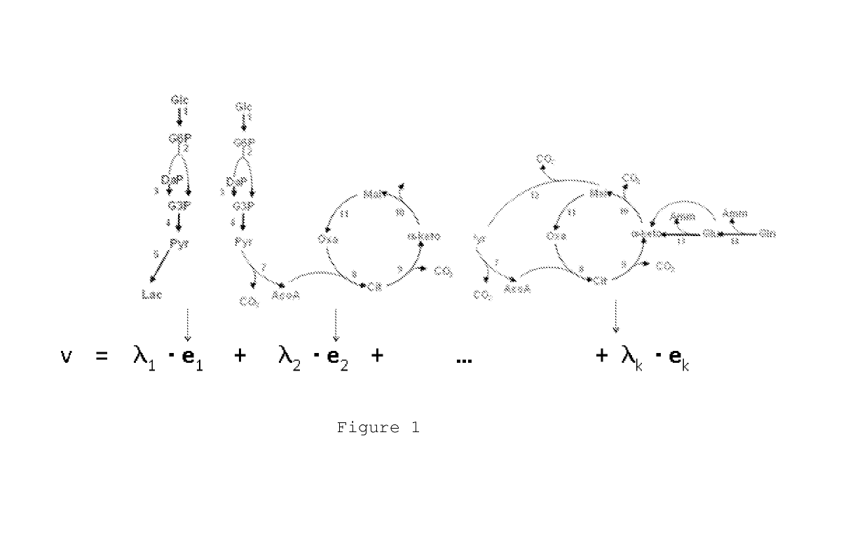

FIG. 2 is a schematic representation of the main steps of the method of the present invention, in which step 1 A—represents the set of cell cultures, B—represents initial and endpoint exometabolome assays; step 3 represents the functional enviromics map, wherein in the xx axis elementary cellular functions set by the universe of genes are represented and in the yy axis medium factors are represented; step 3 represents the formulation of optimized culture medium and step 4 represents the final validation in cell culture triplicates in optimized medium formulations.

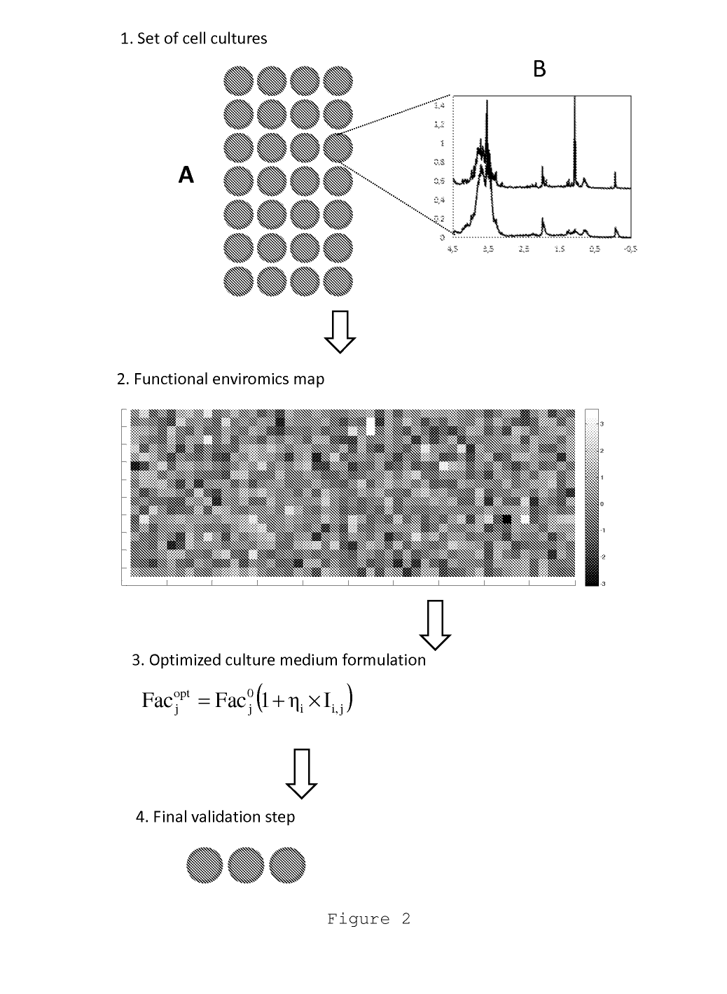

FIG. 3 exemplifies the application of the cell culture protocol of the present invention to a recombinant Pichia pastoris X33 strain. It provides the results of biomass concentration, product concentration and ammonium concentration, after 110 hours of incubation time, for 26 culture experiments.

FIG. 4 shows a low resolution Functional Enviromics map for a recombinant Pichia pastoris X33 strain.

### EXAMPLES

Hereinafter, the present invention is described in more detail and specifically with reference to the Examples, which however are not intended to limit the present invention.

### Example 1

### Elementary Cellular Functions for the Yeast Pichia pastoris X33

Herein the steps for obtaining the working set of elementary cellular functions for the recombinant yeast Pichia pastoris X33 are described.

First, a metabolic network was built from literature sources, namely the KEGG database [30] and papers by Chung et al. [31] and [symbol] elik et al. [32]. The genes associated to each reaction are in most cases known and can be found in Chung et al. [31]. The metabolic network was further simplified by lumping together in single reactions the anabolic pathways. The resulting metabolic network has 99 reactions (thus 99 fluxes), 89 intracellular metabolites and 9 extracellular metabolites. The complete set of metabolic reactions are listed in Table 1.

TABLE 1
List of metabolic reactions of a recombinant Pichia
pastoris X33 strain expressing a protein of empirical formula
CH_{1.965}N_{0.271}O_{0.535}S_{0.006}
Uptake reactions

| | |
|---|---|
| R1 | 1 ATP + 1 GlyOH → 1 ADP + 1 GAP + 1 NADH₂ |
| R2 | 2 ATP + 1 H₃PO₄ + 2 H₂O → 2 ADP + 3 Pi |
| R3 | 2 ATP + 4 NADH₂ + 1 H₂SO₄ → 2 ADP + 1 PPi + 3 H₂O + 1 H₂S |

Glycolysis/Glyconeogenesis

| | |
|---|---|
| R4 | 1 G6P [symbol] 1 F6P |
| R5 | 1 ATP + 1 F6P → 1 ADP + 2 GAP |
| R6 | 1 H₂O + 2 GAP → 1 F6P + 1 Pi |
| R7 | 1 ADP + 1 Pi + 1 GAP [symbol] 1 ATP + 1 PEP + 1 H₂O + 1 NADH₂ |
| R8 | 1 ADP + 1 PEP → 1 Pyr + 1 ATP |

Pentose Phosphate Pathway

| | |
|---|---|
| R9 | 1 G6P + 1 H₂O → 1 R5P +2 NADH₂ + 1 CO₂ |
| R10 | 1 R5P [symbol] 1 X5P |
| R11 | 1 R5P + 1 X5P [symbol] E4P + 1 F6P |
| R12 | 1 E4P + 1 X5P [symbol] 1 GAP + 1 F6P |
| R13 | 1 ATP + 1 R5P → 1 AMP* + 1 PRPP |

Anaplerotic reactions

| | |
|---|---|
| R14 | 1 Pyr + 1 H₂O + 1 CO₂ + 1 ATP → 1 OA + 1 ADP + 1 Pi |
| R15 | 1 Mal → 1 Pyr + 1 CO₂ + 1 NADH₂ |
| R16 | 1 OA + 1 ATP [symbol] 1 PEP + 1 ADP + 1 CO₂ |

Tricarboxylic acid (TCA) cycle

| | |
|---|---|
| R17 | 1 OA + 1 AcCoA + 1 H₂O → 1 Cit |
| R18 | 1 Cit → 1 ICit |
| R19 | 1 ICit [symbol] 1 aKG +1 NADH₂ + 1 CO₂ |
| R20 | 1 ADP + 1 Pi + 1 aKG → 1 ATP + 1 NADH₂ + 1 CO₂ + 1 Succ |
| R21 | 1 Succ → 1 Fum +1 NADH₂ |
| R22 | 1 Fum + 1 H₂O [symbol] 1 Mal |
| R23 | 1 Mal [symbol] 1 OA + 1 NADH₂ |

Glyoxylate Shunt

| | |
|---|---|
| R24 | 1 ICit → 1 Glx + 1 Succ |
| R25 | 1 AcCoA + 1 Glx + 1 H₂O → 1 Mal |

C3/C4 Metabolism pathway

| | |
|---|---|
| R26 | 1 Pyr → 1 AcCoA + 1 CO₂ + 1 NADH₂ |

Proteins Biosynthesis

| | |
|---|---|
| R27 | 1 Asp + 1 Ser + 1 ATP + 1 H₂O + 1 H₂S → 1 Cys + 1 ADP + 1 Pi + |
| | 1 Pyr + 1 NH₃ + 1 CO₂ + 1 NADH₂ |
| R28 | 1 Glu + 1 Gln + 1 Asp + 4 ATP + 1 CO₂ +2 H₂O + 1 NADH₂ → 1 Arg + |
| | 1 aKG + 3 ADP + 1 AMP* +3 Pi + 1 PPi + 1 Fum |
| R29 | 2 Glu + 1 AcCoA → 1 Lys + 1 aKG + 1 CO₂ |
| R30 | 2 Pyr + 1 AcCoA + 1 Glu → 1 Leu + 2 CO₂ + 1 aKG |
| R31 | 1 PRPP + 1 ATP + 3 H₂O + 1 Gln → 1 His + 1 AICAR + 2 NADH₂ + 2 |
| | PPi + 1 Pi + 1 aKG |
| R32 | 1 Glu + 1 ATP + 1 NH₃ → 1 Gln + 1 ADP + 1 Pi |
| R33 | 1 aKG + 1 NADH₂ + 1 NH₃ → 1 Glu + 1 H₂O |
| R34 | 1 Glu + 1 OA → 1 Asp + 1 aKG |
| R35 | 1 Asp + 1 Gln + 1 ATP + 1 H₂O → 1 Asn + 1 Glu + 1 AMP* + 1 PPi |
| R36 | 1 Glu + 1 Pyr → 1 Ala + 1 aKG |
| R37 | 1 E4P + 2 PEP + 1 NADH₂ + 1 ATP → 1 Chor + 1 ADP + 4 Pi |
| R38 | 1 Chor + 1 PRPP + 1 Gln + 1 Ser → 1 Trp + 1 GAP + 1 Glu + 1 CO₂ + 1 H₂O + |
| | 1 Pyr + 2 Pi |
| R39 | 1 Chor + 1 Glu → 1 Tyr + 1 NADH₂ + 1 aKG + 1 CO₂ |
| R40 | 1 Chor + 1 Ala → 1 Tyr + 1 NADH₂ + 1 Pyr + 1 CO₂ |
| R41 | 1 Chor + 1 Glu → 1 Phe + 1 aKG + 1 H₂O + 1 CO₂ |
| R42 | 1 Chor + 1 Ala → 1 Phe + 1 Pyr + 1 H₂O + 1 CO₂ |
| R43 | 1 Glu + 2 Pyr + 1 NADH₂ → 1 Val + 1 H₂O + 1 CO₂ + 1 aKG |
| R44 | 1 Glu + 1 GAP + 2 H₂O → 1 Ser + 1 aKG + 1 Pi + 2 NADH₂ |
| R45 | 1 Gly + 1 MnTHF + 1 H₂O → 1 Ser + 1 THF |
| R46 | 1 Asp + 2 ATP + 2 NADH₂ + 1 H₂O → Thr + 2 ADP + 2 Pi |
| R47 | 1 Asp + 1 ATP + 2 NADH₂ +1 H₂S + 1 MnTHF + 3 NADH₂ → 1 Met + 1 ADP + |
| | 1 Pi + 1 THF + 1 H₂O |
| R48 | 1 Ser + 1 THF → 1 Gly + 1 MnTHF + 1 H₂O |
| R49 | 1 Glx + 1 Ala → 1 Gly + 1 Pyr |
| R50 | 1 Glu + 1 ATP + 2 NADH₂ → 1 Pro + 1 ADP + 1 Pi + 1 H₂O |
| R51 | 1 Glu + 1 Pyr + 1 Thr +1 NADH₂ → 1 aKG + 1 ILeu + 1 H₂O + 1 CO₂ + 1 NH₃ |
| R52 | 0.1245 Ala + 0.0437 Arg + 0.0277 Asn + 0.0808 Asp + 0.0019 Cys + |
| | 0.0285 Gln + 0.0819 Glu + 0.0787 Gly + 0.0179 His + 0.0524 |
| | ILeu + 0.0803 Leu + 0.0776 Lys + 0.0138 Met + 0.0364 Phe + 0.0448 Pro + |
| | 0.0502 Ser + 0.0518 Thr + 0.0076 Trp + 0.0277 Tyr + 0.0719 Val + 4.3 ATP + |
| | 4.3 H₂O => 1 Prot + 4.3 Pi + 4.3 ADP |

Lipids Biosynthesis

| | |
|---|---|
| R53 | 9 AcCoA + 8 ATP + 1 GAP + 17 NADH₂ + 1 H₂O 8 ADP + 9 Pi + 1 MAG |
| R54 | 18 AcCoA + 16 ATP + 1 GAP + 34 NADH₂ + 1 H₂O → 16 ADP + 17 Pi + 1 DAG |
| R55 | 27 AcCoA + 24 ATP + 1 GAP + 51 NADH₂ + 1 H₂O → 24 ADP + 25 Pi + |
| | 1 TAG |
| R56 | 18 AcCoA +18 ATP + 23 NADH₂ + 10 O₂ → 18 ADP + 6 PPi + 6 Pi + |
| | 1 Zym + 7 H₂O + 9 CO₂ |
| R57 | 1 Met + 1 Zym + 2 O₂ + 1 NADH₂ → 1 Asp + 1 Erg + 2 H₂O + 1 H₂S |
| R58 | 1 Ser + 18 AcCoA + 16 ATP + 1 GAP + 1 CTP + 37 NADH₂ => 16 ADP + |
| | 1 PPi + 16 Pi + 1 CMP* + 1 PDS |
| R59 | 1 PDS → 1 PDE + 1 CO₂ + 3 NADH₂ |
| R60 | 1 PDE + 3 Met + 6 H₂O → 3 Asp + 3 H₂S + 1 PDC + 6 NADH₂ |
| R61 | 1 G6P + 18 AcCoA +16 ATP + 1 GAP + 1 CTP + 34 NADH₂ + 1 H₂O → |
| | 16 ADP + 1 PPi + 17 Pi + 1 CMP* + 1 PDMI |
| R62 | 0.0548 MAG + 0.0548 DAG + 0.0548 TAG + 0.1233 PDC + 0.0411 PDS + |
| | 0.0959 PDE + 0.1507 Erg + 0.3151 Zym + 0.1096 PDMI → 1 Lip |

Carbohydrates Biosynthesis

| | |
|---|---|
| R63 | 1 F6P + H₂O => 1 Pi + 1 Man |
| R64 | 1 G6P + 1 UTP + 1 H₂O => 1 Glc + 1 PPi + 1 UDP |
| R65 | 1 Glc => 1 Gal |
| R66 | 1 Gln + 1 AcCoA + 1 F6P + 1 UTP => 1 Glu + 1 PPi + 1 GlcNAc + 1 UDP |
| R67 | 1 PEP + 1 CTP +1 GlcNAc + 3 H₂O + 1 ATP => 1 PPi + 2 Pi + 1 NeuAc + |
| | 1 CMP* + 1 ADP |
| R68 | 0.1554 NeuAc + 0.1554 GlcNAc + 0.3250 Man + 0.2088 Glc + 0.1554 Gal → 1 Carb |

Nucleotides Biosynthesis

| | |
|---|---|
| R69 | 1 Asp + 2 Gln + 1 Gly + 5 ATP + 1 PRPP + 1 FTHF + 1 CO₂ + 3 H₂O → 2 Glu + |
| | 1 THF + 5 ADP + 5 Pi + 1 PPi + 1 Fum + 1 AICAR |
| R70 | 1 AICAR + 1 FTHF → 1 THF + 1 IMP +1 H₂O |
| R71 | 1 IMP + 1 Asp + 1 GTP → 1 AMP + 1 Fum + 1 Pi + 1 GDP |
| R72 | 1 IMP + 1 Gln + ATP + 2 H₂O → 1 GMP + 1 Glu + 1 PPi + AMP* + 1 |
| | NADH₂ |
| R73 | 1 IMP + 1 NH₃ + ATP + 1 H₂O → 1 GMP + 1 PPi + AMP* |
| R74 | 1 PRPP + 1 Asp + 1 Gln + 2 ATP + 1 H₂O → 1 UMP + 1 Glu + 1 |
| | NADH₂ + 1 PPi + 2 Pi + 2 ADP |
| R75 | 1 NH₃ + 1 UMP → 1 CMP + H₂O |
| R76 | 1 CMP + 1 NADH₂ → 1 dCMP + H₂O |
| R77 | 1 GMP + 1 NADH₂ → 1 dGMP + H₂O |
| R78 | 1 AMP + 1 NADH₂ → 1 dAMP + H₂O |
| R79 | 1 UMP + MnTHF + 2 NADH₂ → dTMP + THF + H₂O |
| R80 | 0.2329 AMP + 0.3059 UMP + 0.2329 GMP + 0.2283 CMP + 0.4 ATP + |
| | 0.4 H₂O → 1 RNA + 0.4 Pi + 0.4 ADP |
| R81 | 0.2000 dCMP + 0.3000 dTMP + 0.3000 dAMP + 0.2000 dGMP + 0.4 ATP + |
| | 0.4 H₂O → 1 DNA + 0.4 Pi + 0.4 ADP |
| R82 | 0.9471 RNA + 0.0529 DNA → 1 Nuc |

Biomass Synthesis

| | |
|---|---|
| R83 | 0.5716 Prot + 0.0359 Nuc + 0.3855 Sug + 0.0073 Lip → 1 X |

Product Biosynthesis

| | |
|---|---|
| R84 | Ammino acids + 4.3 ATP + 4.3 H2O => Product + 4.3 Pi + 4.3 ADP |

Oxidative Phosphorylation (P/O ratio = 2)

| | |
|---|---|
| R85 | 2 ADP + 2 Pi + 1 NADH₂ + 0.5 O₂ → 2 ATP + 3 H₂O |

Biosynthesis and Interconversion of one-carbon units

| | |
|---|---|
| R86 | 1 MnTHF + 1 H₂O → 1 FTHF + 1 NADH₂ |
| R87 | 1 FTHF + 1 AICAR + 1 OA + 1 NH₃ +1 ATP + 1 GTP + 1 NADH₂ → 1 THF + 2 Pi + |
| | 1 GDP + 1 Fum + 1 AMP* + 1 ADP + 1 H₂O |
| R88 | 1 MnTHF + 2 H₂O → 1 THF + 1 NADH₂ + 1 For |
| R89 | 1 THF + 1 Gly → 1 MnTHF + 1 CO₂ + 1 NH₃ + 1 NADH₂ |
| R90 | 1 For → 1 NADH₂ + 1 CO₂ |

Energy Interconversion

| | |
|---|---|
| R91 | 1 PPi + 1 H₂O → 2 Pi |
| R92 | 1 ATP + 1 AMP* → 2 ADP |
| R93 | 1 ATP + 1 CMP* → 1 CDP + 1 ADP |
| R94 | 1 ATP + 1 CDP → 1 CTP + 1 ADP |
| R95 | 1 ATP + 1 GMP* → 1 GDP + 1 ADP |
| R96 | 1 ATP + 1 GDP → 1 GTP + 1 ADP |
| R97 | 1 ATP + 1 UMP* → 1 UDP + 1 ADP |
| R98 | 1 ATP + 1 UDP → 1 UTP + 1 ADP |
| R99 | 1 ATP + 1 H₂O → 1 ADP + 1 Pi |

The open source bioinformatics software METATOOL 5.0[33] was used to compute the elementary flux modes of the metabolic network specified in Table 1. The total number of elementary flux modes was 3368. In the lines below it is specified five elementary flux modes obtained with METATOOL. 5.0 [30]. Note that the dimension of each elementary flux mode vector is 99 and that the values within it represent the weight of a given metabolic reaction for that particular elementary flux mode.

Elementary mode 9 and 10 correspond to biomass synthesis, elementary mode 3 and 12 correspond to product synthesis and elementary mode 5 corresponds to catabolism. These elementary flux modes were found to be the most important in posterior steps of the method of the present invention and this is the reason we specify then here:

$${e}_{10} = [ \begin{matrix}7.22 \; 0.13 \; 0.03 - 0.46 \; 6.19 \; 7.59 \; 3.95 \; 3.49 \; 0.00 \\ - 0.15 - 0.01 - 0.14 \; 0.16 \; 1.19 \; 0.20 \; 0.00 \; 0.51 \; 0.51 \; 0.51 \\ 0.00 \; 0.00 \; 0.20 \; 0.00 \; 0.00 \; 0.00 \; 1.62 \; 0.00 \; 0.08 \; 0.14 \; 0.15 \; 0.03 \; 0.82 \\ 2.46 \; 0.65 \; 0.05 \; 0.30 \; 0.13 \; 0.01 \; 0.05 \; 0.00 \; 0.00 \; 0.07 \; 0.13 \; 0.32 \; 0.00 \\ 0.19 \; 0.04 \; 0.20 \; 0.00 \; 0.08 \; 0.10 \; 1.85 \; 0.00 \; 0.00 \; 0.00 \; 0.01 \; 0.00 \; 0.01 \\ 0.01 \; 0.00 \; 0.00 \; 0.02 \; 0.41 \; 0.45 \; 0.19 \; 0.39 \; 0.19 \; 1.25 \; 0.05 \; 0.05 \; 0.03 \\ 0.03 \; 0.00 \; 0.06 \; 0.03 \; 0.00 \; 0.00 \; 0.00 \; 0.00 \; 0.11 \; 0.01 \; 0.12 \; 3.24 \\ 0.00 \; 10.11 \; 0.14 \; 0.03 \; 0.02 \; 0.00 \; 0.02 \; 1.48 \; 0.36 \; 0.20 \; 0.20 \; 0.00 \\ 0.06 \; 0.00 \; 0.84 \; 0.00\end{matrix} ] ; \\ {e}_{12} = [ \begin{matrix}9.00 \; 0.00 \; 0.15 \; 0.00 \; 1.94 \; 2.18 \; 6.50 \; 5.36 \; 0.00 - 0.25 \; 0.16 \\ - 0.41 \; 0.08 \; 1.67 \; 0.11 \; 0.25 \; 0.15 \; 0.40 \; 0.00 \; 0.67 \; 6.05 \; 1.20 \\ 0.06 \; 0.34 \; 0.57 \; 0.08 \; 0.25 \; 0.00 \; 0.23 \; 0.00 \; 0.25 \; 1.69 \; 0.00 \\ 0.57 \; 0.04 \; 0.70 \; 0.00 \; 0.25 \; 0.17 \; 0.00 \; 0.00 \; 0.00 \; 0.00 \; 0.00 \; 0.00 \\ 0.00 \; 0.00 \; 0.00 \; 0.00 \; 0.00 \; 0.00 \; 0.00 \; 0.00 \; 0.00 \; 0.00 \; 0.00 \; 0.00 \\ 0.00 \; 0.00 \; 0.00 \; 0.00 \; 0.00 \; 0.00 \; 0.00 \; 0.00 \; 0.00 \; 0.00 \; 0.00 \; 0.00 \\ 0.00 \; 0.00 \; 5.12 \; 13.91 \; 0.00 \; 0.00 \; 0.00 \; 0.65 \; 0.00 \; 0.65 \; 0.46 \\ 0.40 \; 0.00 \; 0.00 \; 0.00 \; 0.00 \; 0.00 \; 0.00 \; 0.00\end{matrix} ] ; \\ {e}_{5} = [ \begin{matrix}1.08 \; 0.00 \; 0.00 - 3.23 \; 0.00 \; 1.08 \; 0.00 \; 14.00 \; 3.23 \; 2.15 \; 1.08 \\ 1.08 \; 0.00 \; 14.00 \; 0.00 \; 14.00 \; 0.00 \; 0.00 \; 0.00 \; 0.00 \; 0.00 \; 0.00 \\ 0.00 \; 0.00 \; 0.00 \; 0.00 \; 0.00 \; 0.00 \; 0.00 \; 0.00 \; 0.00 \; 0.00 \; 0.00 \; 0.00 \\ 0.00 \; 0.00 \; 0.00 \; 0.00 \; 0.00 \; 0.00 \; 0.00 \; 0.00 \; 0.00 \; 0.00 \; 0.00 \; 0.00 \\ 0.00 \; 0.00 \; 0.00 \; 0.00 \; 0.00 \; 0.00 \; 0.00 \; 0.00 \; 0.00 \; 0.00 \; 0.00 \; 0.00 \\ 0.00 \; 0.00 \; 0.00 \; 0.00 \; 0.00 \; 0.00 \; 0.00 \; 0.00 \; 0.00 \; 0.00 \; 0.00 \; 0.00 \\ 0.00 \; 0.00 \; 0.00 \; 0.00 \; 0.00 \; 0.00 \; 0.00 \; 0.00 \; 0.00 \; 0.00 \; 0.00 \; 0.00 \\ 0.00 \; 0.00 \; 7.54 \; 0.00 \; 0.00 \; 0.00 \; 0.00 \; 0.00 \; 0.00 \; 0.00 \; 0.00 \; 0.00 \\ 0.00 \; 0.00 \; 0.00 \; 0.00 \; 0.00\end{matrix} ] ; \\ {e}_{9} = [ \begin{matrix}47.58 \; 0.82 \; 0.19 - 4.48 \; 0.00 \; 9.66 \; 25.76 \; 22.76 \; 1.50 \; 0.00 \; 0.43 \\ - 0.43 \; 1.07 \; 7.78 \; 1.28 \; 0.00 \; 3.34 \; 3.34 \; 3.34 \; 0.00 \; 0.00 \; 1.28 \; 0.00 \\ 0.00 \; 0.00 \; 10.56 \; 0.02 \; 0.53 \; 0.94 \; 0.97 \; 0.22 \; 5.16 \; 16.07 \; 4.23 \\ 0.33 \; 2.28 \; 0.87 \; 0.09 \; 0.00 \; 0.33 \; 0.00 \; 0.44 \; 0.87 \; 2.07 \; 0.00 \; 1.26 \\ 0.25 \; 1.31 \; 0.00 \; 0.54 \; 0.63 \; 12.08 \; 0.01 \; 0.01 \; 0.01 \; 0.07 \; 0.02 \; 0.04 \\ 0.03 \; 0.02 \; 0.02 \; 0.15 \; 2.65 \; 2.97 \; 1.27 \; 2.53 \; 1.27 \; 8.15 \; 0.35 \; 0.35 \\ 0.18 \; 0.00 \; 0.18 \; 0.40 \; 0.17 \; 0.01 \; 0.01 \; 0.01 \; 0.01 \; 0.72 \; 0.04 \; 0.76 \\ 21.14 \; 0.00 \; 69.30 \; 0.93 \; 0.22 \; 0.12 \; 0.00 \; 0.12 \; 9.67 \; 2.32 \; 1.32 \\ 1.32 \; 0.00 \; 0.40 \; 0.00 \; 5.50 \; 46.69\end{matrix} ] ; \\ {e}_{3} = [ \begin{matrix}37.03 \; 0.00 \; 0.60 - 1.50 \; 0.00 \; 1.50 \; 26.37 \; 21.74 \; 1.50 \; 0.00 \; 1.16 \\ - 1.16 \; 0.34 \; 9.42 \; 1.03 \; 0.00 \; 4.54 \; 4.54 \; 4.54 \; 0.00 \; 0.00 \; 1.03 \; 0.00 \\ 0.00 \; 2.31 \; 0.34 \; 0.00 \; 1.03 \; 0.00 \; 0.94 \; 1.03 \; 6.85 \; 0.00 \; 2.31 \; 0.17 \\ 2.83 \; 0.00 \; 1.03 \; 0.68 \; 0.00 \; 0.00 \; 0.00 \; 0.00 \; 0.00 \; 0.00 \; 0.00 \; 0.00 \\ 0.00 \; 0.00 \; 0.00 \; 0.00 \; 0.00 \; 0.00 \; 0.00 \; 0.00 \; 0.00 \; 0.00 \; 0.00 \; 0.00 \\ 0.00 \; 0.00 \; 0.00 \; 0.00 \; 0.00 \; 0.00 \; 0.00 \; 0.00 \; 0.00 \; 0.00 \; 0.00 \; 0.00 \\ 20.80 \; 59.99 \; 0.00 \; 0.00 \; 2.65 \; 0.00 \; 2.65 \; 1.88 \; 1.63 \; 0.00 \; 0.00 \; 0.00 \\ 0.00 \; 0.00 \; 0.00 \; 13.34\end{matrix} ] ;$$

### Example 2

### Array of 24 Culture Experiments for Screening N=11 Medium Factors for the Yeast Pichia pastoris X33

Here it is described how the screening experiments are performed for the yeast Pichia pastoris X33.

Eleven medium factors (N=11) were selected for screening (listed in Table II). Each medium factor as a baseline value taken from the Invitrogen guidelines [34], a −1 level (10 times lower than the baseline value) and a +1 level, coincident to the baseline value

An array of 24=2×(N+1) cell cultures plus 2 control experiments were performed in 250 ml T-flasks with varying medium composition. The combinations of medium factor values to be tested in each experiment are listed in Table III. These were obtained by a D-optimal design for linear function identification using 24 independent experiments.

TABLE II
List of medium factors, respective baseline values and upper
(+1) and lower (−1) values for cell culture experiments.
The final medium formulation comprises mixtures of 1:200
(v/v) of the PTM1 and diluted BSM solutions respectively

| | | | | |
|---|---|---|---|---|
| Medium factors | Units | Baseline | −1 level | +1 level |

PTM1 solution¹

| | | | | | |
|---|---|---|---|---|---|
| Fac₁ | CuSO₄•5H₂O | g/L | 6.00 | 0.6 | 6.00 |
| Fac₂ | NaI | g/L | 0.08 | 0.008 | 0.08 |
| Fac₃ | MnSO₄•H₂O | g/L | 3.00 | 0.3 | 3.00 |
| Fac₄ | Na₂MoO₄•2H₂O | g/L | 0.20 | 0.02 | 0.20 |
| Fac₅ | H₃BO₃ | g/L | 0.02 | 0.002 | 0.02 |
| Fac₆ | CoCl₂•6H₂O | g/L | 0.50 | 0.05 | 0.50 |
| Fac₇ | ZnCl₂ | g/L | 20.00 | 2 | 20.00 |
| Fac₈ | FeSO₄•7H₂O | g/L | 65.00 | 6.5 | 65.00 |
| Fac₉ | Biotin | g/L | 0.20 | 0.02 | 0.20 |
| Fac₁₀ | H₂SO₄ | mL/L | 5.00 | 0.5 | 5.00 |

BSM solution²

| | | | | | |
|---|---|---|---|---|---|
| Fac₁₁ | BSM dilution factor | v/v | 1:1 | 0.5:1 | 1:1 |

¹Pichia Trace Metals salts supplements (PTM1) (see [34])
²The Basal Salts Medium solution composition is: H3PO4 85%, 26.70 ml/L, CaSO4•2H2O 0.93 g/L, K2SO4 18.20 g/L, MgSO4•7H2O 14.90 g/L, KOH 4.13 g/L

TABLE III
D-optimal experimental design for linear function
identification for 11 factors (columns) and 24 experiments
(rows). The +1 and −1 levels are specified in Table I

| | Fac1 | Fac2 | Fac3 | Fac4 | Fac5 | Fac6 | Fac7 | Fac8 | Fac9 | Fac10 | Fac11 |
|---|---|---|---|---|---|---|---|---|---|---|---|
| Exp. 1 | 1 | 1 | −1 | −1 | 1 | −1 | 1 | −1 | −1 | −1 | 1 |
| Exp. 2 | 1 | −1 | −1 | 1 | −1 | −1 | −1 | 1 | −1 | −1 | 1 |
| Exp. 3 | 1 | −1 | 1 | 1 | 1 | −1 | 1 | 1 | 1 | −1 | −1 |
| Exp. 4 | 1 | 1 | −1 | −1 | 1 | 1 | −1 | 1 | 1 | 1 | −1 |
| Exp. 5 | 1 | 1 | 1 | 1 | 1 | −1 | −1 | −1 | 1 | 1 | 1 |
| Exp. 6 | −1 | 1 | −1 | 1 | 1 | 1 | 1 | −1 | 1 | −1 | −1 |
| Exp. 7 | −1 | 1 | 1 | 1 | 1 | 1 | 1 | 1 | 1 | 1 | 1 |
| Exp. 8 | 1 | 1 | 1 | 1 | −1 | 1 | 1 | 1 | −1 | −1 | −1 |
| Exp. 9 | 1 | −1 | −1 | 1 | −1 | −1 | 1 | 1 | 1 | 1 | −1 |
| Exp. 10 | −1 | 1 | 1 | −1 | −1 | −1 | −1 | −1 | 1 | −1 | −1 |
| Exp. 11 | −1 | 1 | −1 | 1 | −1 | 1 | −1 | 1 | −1 | −1 | 1 |
| Exp. 12 | −1 | −1 | −1 | −1 | 1 | −1 | 1 | 1 | 1 | −1 | 1 |
| Exp. 13 | −1 | −1 | 1 | 1 | 1 | 1 | −1 | −1 | −1 | −1 | 1 |
| Exp. 14 | 1 | 1 | −1 | 1 | −1 | 1 | 1 | −1 | 1 | −1 | 1 |
| Exp. 15 | 1 | −1 | −1 | 1 | 1 | 1 | −1 | −1 | −1 | 1 | −1 |
| Exp. 16 | 1 | 1 | 1 | −1 | 1 | −1 | −1 | 1 | −1 | −1 | −1 |
| Exp. 17 | 1 | −1 | 1 | −1 | −1 | 1 | −1 | −1 | 1 | −1 | 1 |
| Exp. 18 | −1 | 1 | 1 | −1 | −1 | 1 | 1 | 1 | −1 | 1 | 1 |
| Exp.19 | −1 | −1 | −1 | −1 | 1 | 1 | 1 | −1 | −1 | −1 | −1 |
| Exp. 20 | −1 | −1 | 1 | 1 | −1 | −1 | 1 | −1 | −1 | 1 | −1 |
| Exp. 21 | −1 | 1 | −1 | 1 | 1 | −1 | −1 | 1 | −1 | 1 | 1 |
| Exp. 22 | −1 | −1 | −1 | −1 | −1 | 1 | −1 | 1 | 1 | 1 | −1 |
| Exp. 23 | 1 | 1 | −1 | −1 | −1 | −1 | 1 | −1 | −1 | 1 | 1 |
| Exp. 24 | 1 | −1 | 1 | −1 | 1 | 1 | 1 | 1 | −1 | 1 | 1 |
| Exp. 25¹ | 1 | 1 | 1 | 1 | 1 | 1 | 1 | 1 | 1 | 1 | 1 |
| Exp. 26¹ | −1 | −1 | −1 | −1 | −1 | −1 | −1 | −1 | −1 | −1 | −1 |

(1) control experiments

Each medium with compositions specified in tables II and III were formulated according to the following experimental procedure. 40 ml of diluted BSM solution (see Table II) was supplemented with glycerol (concentration of 20 g/L) and autoclaved at 121° C. for 30 minutes. The pH of BSM is approximately 1.5 and must be adjusted to the working pH of 5.0 with addition of 25% ammonium hydroxide after autoclaving. The ammonium hydroxide also serves as nitrogen source. The PTM1 trace salts stock solution was first sterilized by filtration with 0.22 nm pore size filter and then added to the autoclaved BSM solution at a ratio of 1:200 (v/v) PTM1:BSM.

The cryogenic vials containing the cells were stored at −80° C. The pre-inoculum was prepared with 1 ml of cell stock and 40 ml of medium with baseline composition (Table II) and then incubated at 30° C. and 150 rpm on an Innova 4300 incubator shaker. When the pre-inoculum achieved exponential growth, approximately after 26 h of incubation time, 2 mL of it was used to inoculate all the 26 T-flasks in parallel.

Each of the 26 T-flasks were incubated for 110 hours at 30° C. and 150 rpm on an Innova 4300 incubator shaker. Samples were taken at intervals of 24 hours. Each sample was analyzed for the following compounds
Biomass concentration was determined by optical density at 600 nm (OD600) and by the wet cell weight method (centrifugation at 15000 rpm followed by weighting without drying).
Product concentration was determined by the Enzyme-Linked ImmunoSorbent Assay (ELISA).
The concentration in the supernatant of glycerol and several organic acids (malic acid, succinic acid, lactic acid, formic acid, fumaric acid) were determined by high performance liquid chromatography (HPLC).
Ammonium concentration was measured using an electrode (Thermo Electron Corporation, Orion 9512).

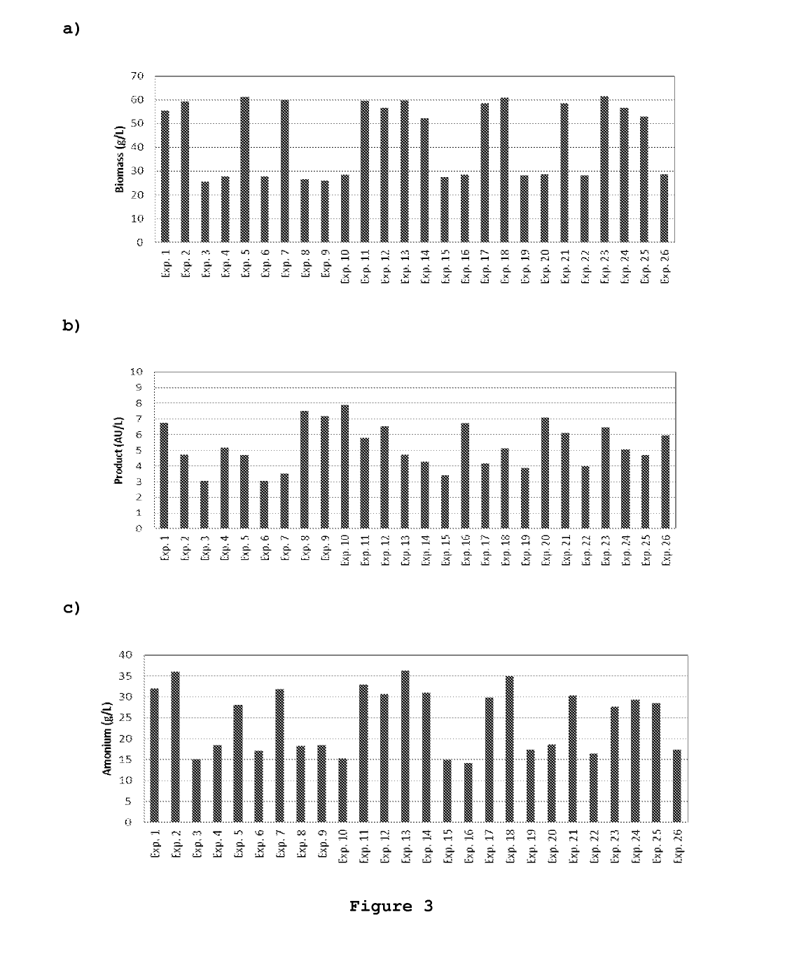

FIGS. 3a-c show the Biomass concentration, product concentration and ammonium concentration in the end of the 26 experiments. The glycerol and organic acids concentrations are shown in Table IV. Organic acids compounds showed residual concentrations. Glycerol was completely exhausted in all cases.

TABLE IV
Exometabolome data (glycerol and organic acids
concentrations) determined by HPLC at time 110 hours

| | | Malic | Succinic | Lactic | Formic | Fumaric |
|---|---|---|---|---|---|---|
| | Gly | Acid | Acid | Acid | Acid | Acid |
| | (g/L) | (g/L) | (g/L) | (g/L) | (g/L) | (g/L) |
| Exp. 1 | 0.12 | 0.28 | 0.46 | 0.12 | 0.34 | 0.08 |
| Exp. 8 | 0.01 | 0.15 | 0.11 | 0.10 | 0.11 | 0.07 |
| Exp. 10 | 0.03 | 0.11 | 0.08 | 0.06 | 0.09 | 0.08 |
| Exp. 20 | 0.01 | 0.07 | 0.12 | 0.10 | 0.12 | 0.13 |
| Exp. 25 | 0.07 | 0.24 | 0.26 | 0.11 | 0.09 | 0.11 |
| Exp. 26 | 0.01 | 0.19 | 0.09 | 0.09 | 0.15 | 0.07 |

### Example 3

### Functional Enviromics Map of Pichia pastoris X33

Here it is exemplified how the experimental data collected in example 2 can be processed into the form of a functional Enviromics map for the yeast Pichia pastoris X33.

Firstly, the rate of change of each compound is calculated by the following formula:

$${v}_{i}^{k} = \cdot \frac{{C}_{Mi}^{k} ( {t}_{batch} ) - {C}_{Mi}^{k} ( 0 )}{{C}_{X}^{av} {t}_{batch}} \qquad ( 10 )$$

with C_{Mi}(0) the initial concentration, C_{Mi}(t_{batch}) the endpoint concentration, Xₐᵥ the average cellular concentration, t_{batch} the duration of the batch experiment. Note that the superscript index denotes experiment while the subscript index denotes compound.

Then perform a statistical regression analysis of v against medium factor values using the following linear model:

$$\begin{matrix}v = \cdot {\sum }_{i = 1}^{K} {λ}_{i} \times {e}_{i} & ( 1 ) \\ {λ}_{i} = \cdot {\sum }_{j = 1}^{N} {I}_{j , i} \times {FAC}_{j} & ( 4 )\end{matrix}$$

Finally organize the data in the form of a functional Enviromics map by putting the intensity values into the form of a N×K array:

Functional Enviromics map={I_{j,i}}

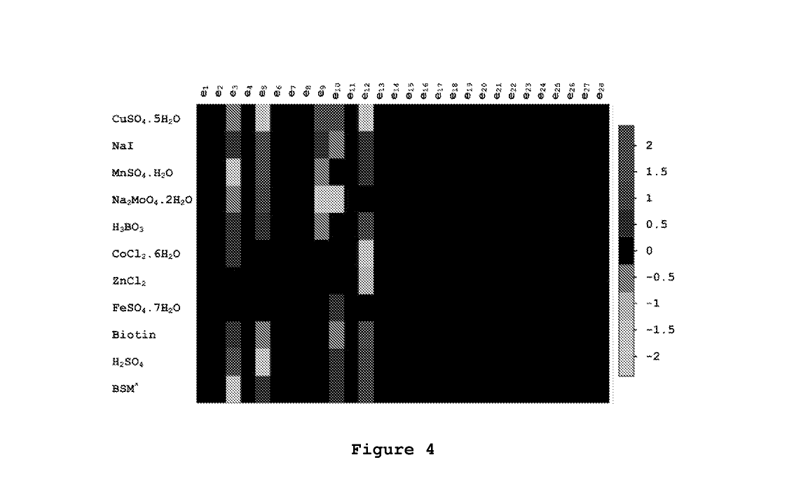

The end result of this procedure is shown in Table V and represented in FIG. 4.

TABLE V
Functional Enviromics map for the Pichia pastoris X33. Only
the most significant elementary cellular functions are shown. All
intensity values below 1% of the standard deviation were removed.
A graphical representation of this data is shown in FIG. 4

| | e10 | e12 | e5 | e9 | e3 |
|---|---|---|---|---|---|
| CuSO₄•5H₂O | 0.06 | 0.13 | 0.09 | −0.06 | −0.05 |
| NaI | 0.04 | 0.18 | −0.16 | 0.00 | 0.04 |
| MnSO₄•H₂O | −0.16 | 0.21 | 0.00 | −0.03 | 0.00 |
| Na₂MoO₄•2H₂O | 0.07 | 0.04 | 0.05 | −0.03 | −0.01 |
| H₃BO₃ | 0.01 | −0.15 | 0.02 | −0.02 | −0.02 |
| CoCl₂•6H₂O | −0.02 | −0.21 | 0.06 | −0.02 | 0.01 |
| ZnCl₂ | −0.02 | 0.15 | 0.11 | −0.10 | 0.00 |
| FeSO₄•7H₂O | −0.76 | 0.01 | 0.19 | −0.32 | −0.02 |
| Biotin | −0.04 | 0.06 | 0.25 | −0.07 | −0.05 |
| H₂SO₄ | −0.20 | 0.06 | 0.24 | 0.05 | 0.00 |
| BSM* | 0.43 | −0.24 | −0.30 | 0.27 | −0.02 |

### Example 4

### Optimized Culture Media Formulation to Enhance the Product Elementary Function by an Enhancement Factor of 60%

Herein it is shown how optimized media formulations can be obtained with much higher product specific productivity than that of baseline medium formulation using the information contained in the functional Enviromics map.

First, iterative adjustments of medium factor values are performed, using the formula (6) so that a target specific productivity of v_{product}=0.16 μg/g dry cell weight/day which corresponds to 60% improvement in relation to the baseline experiment.

FACⱼ^{opt}=(1+ηᵢIᵢⱼ)FACⱼ⁽⁰⁾ (9)

This procedure resulted in the following optimized medium composition

Optimized Formulation

CuSO₄.5H₂O, 7.25 g/L, NaI, 0.106 g/L, MnSO₄.H₂O, 1.152 g/L, Na₂MoO₄.2H₂O, 0.036 g/L, H₃BO₃, 0.013 g/L, CoCl₂.6H₂O, 0.25 g/L, ZnCl₂, 25.2 g/L, FeSO₄.7H₂O, 5.34 g/L, Biotine, 0.038 g/L, H₂SO₄, 1.989 mL/L, diluição de BSM, 0.84:1 (v/v)

T-flask culture experiments were executed either using the baseline medium formulation (control experiment specified in Table II) or the optimized medium formulation. The applied protocol was the one described in example 2. The final measured specific productivities were the following:
Baseline formulation: 0.10 μg/g dry cell weight/day
Optimized formulation: 0.17 μg/g dry cell weight/day

An enhancement factor of 62.3% was obtained in relation to the baseline formulation.

### Example 5

### Optimized Culture Media Formulation to Enhance the Product Elementary Function by an Enhancement Factor of 100%

A similar procedure to example 4 was executed but targeting an even higher specific productivity of v_{product}=0.20 μg/g dry cell weight/day which corresponds to 100% improvement in relation to the baseline experiment. The obtained optimized medium formulation was the following:

Optimized Formulation

CuSO₄. 5H₂O, 12.0 g/L, NaI, 0.16 g/L, MnSO₄.H₂O, 6.00 g/L, Na₂MoO₄.2H₂O, 0.40 g/L, H₃BO₃, 0.001 g/L, CoCl₂.6H₂O, 0.25 g/L, ZnCl₂, 40.0 g/L, FeSO₄.7H₂O, 3.25 g/L, Biotina, 0.4 g/L, H₂SO₄, 10.0 mL/L, diluição de BSM, 0.25:1 (v/v)

T-flask culture experiments were executed either using the baseline medium formulation or the optimized medium formulation in a similar way to example 4. The final measured specific productivities were the following:
Baseline formulation: 0.10 μg/g dry cell weight/day
Optimized formulation: 0.22 μg/g dry cell weight/day

An enhancement factor of 104.7% was obtained in relation to the baseline formulation.

### Example 6

### Increase of Recombinant Protein Production in a Pilot Scale 50 Liter Bioreactor by the Yeast Pichia pastoris X33

The same Pichia pastoris X33 strain of previous examples was used in this example. The strain was cultivated in a pilot 50 liter reactor. Two pilot 50 liter experiments were performed either using the baseline medium formulation (specified in Table II) or the optimized medium formulation of example 5, comprising:

CuSO₄.5H₂O, 12.0 g/L, NaI, 0.16 g/L, MnSO₄.H₂O, 6.00 g/L, Na₂MoO₄.2H₂O, 0.40 g/L, H₃BO₃, 0.001 g/L, CoCl₂.6H₂O, 0.25 g/L, ZnCl₂, 40.0 g/L, FeSO₄.7H₂O, 3.25 g/L, Biotine, 0.4 g/L, H₂SO₄, 10.0 mL/L, diluted BSM solution, 0.25:1 (v/v)

For the optimized formulation, the diluted 0.25:1 (v/v) BSM solution was sterilized at 121° C. for 30 minutes; then the PTM1 trace salts stock optimized solution with the same composition of example 5 was added.

For the baseline formulation, the undiluted BSM solution was sterilized at 121° C. for 30 minutes; then the PTM1 trace salts stock solution with the composition specified in table II was added.

All subsequent steps are the same for both baseline and optimized medium formulations, as follows.

A shake flask containing 40 ml of sterilized medium as described above was inoculated with one cryovial from the Pichia pastoris cell stock, and incubated at 30° C. for 3 days, agitated at 150 rpm; 10 ml of this pre-inoculum was used to inoculate a shake flask with 750 ml of optimized medium. This inoculum was grown for 3 days at 30° C., 150 rpm, (OD600 nm=2-6), and used to inoculate the bioreactor. The reactor was inoculated at a starting volume of 15 of sterilized medium. The reactor is operated for approximately 30 hours in batch mode and after 100 hours in glycerol fed-batch. Cultivation temperature was controlled at 30° C. and pH was controlled at 5.0 with addition of ammonium hydroxide 25%. In Glycerol fed-batch the dissolved oxygen is kept constant at a low level (5% of saturation) by closed-loop manipulation of the glycerol feeding rate. The final product production, at 90 hours cultivation, were the following for both experiments:
Pilot 50 liter experiment with baseline medium formulation: final protein titer 219.32 mg/L, final protein yield 0.27 mg protein/g wet cell weight
Pilot 50 liter experiment with optimized medium formulation: final protein titer 258.04 mg/L, final protein yield 0.38 mg protein/g wet cell weight

An enhancement factor of 18% in the final product titer and of 38% in the final protein yield was obtained with the optimized medium formulation in relation to the baseline formulation.

### REFERENCES

1. Dong, J., et al., Evaluation and optimization of hepatocyte culture media factors by design of experiments (DoE) methodology. Cytotechnology, 2008. 57(3): p. 251-261.
2. Mandenius, C. F. and A. Brundin, Bioprocess Optimization Using Design-of-Experiments Methodology. Biotechnology Progress, 2008. 24(6): p. 1191-1203.
3. Hunter, D. J., Gene-environment interactions in human diseases. Nature Reviews Genetics, 2005. 6(4): p. 287-298.
4. Cunha, A. E., et al., Methanol induction optimization for scFv antibody fragment production in Pichia pastoris. Biotechnology and Bioengineering, 2004. 86(4): p. 458-467.
5. Deshpande, R. R., C. Wittmann, and E. Heinzle, Microplates with integrated oxygen sensing for medium optimization in animal cell culture. Cytotechnology, 2004. 46(1): p. 1-8.
6. Chang, K. H. and P. W. Zandstra, Quantitative screening of embryonic stem cell differentiation: Endoderm formation as a model. Biotechnology and Bioengineering, 2004. 88(3): p. 287-298.
7. Montgomery D. C., 2005, Design and Analysis of Experiments, John Wiley & Sons Inc, 6^{th} edition
8. Ghosalkar, A., V. Sahai, and A. Srivastava, Optimization of chemically defined medium for recombinant Pichia pastoris for biomass production. Bioresource Technology, 2008. 99(16): p. 7906-7910.
9. Kell, D. B., et al., Metabolic footprinting and systems biology: The medium is the message. Nature Reviews Microbiology, 2005. 3(7): p. 557-565.
10. Schuster, S, and H. Claus, On Elementary Flux Modes in Biochemical Reaction Systems at Steady State. Journal of Biological Systems, 1994. 2(2): p. 165-182.
11. Schuster, S., T. Dandekar, and D. A. Fell, Detection of elementary flux modes in biochemical networks: a promising tool for pathway analysis and metabolic engineering. Trends in Biotechnology, 1999. 17(2): p. 53-60.
12. Schuster, S., D. A. Fell, and T. Dandekar, A general definition of metabolic pathways useful for systematic organization and analysis of complex metabolic networks. Nature Biotechnology, 2000. 18(3): p. 326-332.
13. Klamt, S, and J. Stelling, Two approaches for metabolic pathway analysis? Trends in Biotechnology, 2003. 21(2): p. 64-69.
14. Palsson, B. O., N. D. Price, and J. A. Papin, Development of network-based pathway definitions: the need to analyze real metabolic networks. Trends in Biotechnology, 2003. 21(5): p. 195-198.
15. Papin, J. A., et al., Comparison of network-based pathway analysis methods. Trends in Biotechnology, 2004. 22(8): p. 400-405.
16. Wagner, C., Nullspace approach to determine the elementary modes of chemical reaction systems. Journal of Physical Chemistry B, 2004. 108(7): p. 2425-2431.
17. http://pinguin.biologie.uni-jena.de/bioinformatik/networks/metatool/metatool5.0/metatool5.0.html
18. http://www.brenda-enzymes.org/
19. http://www.pichiagenome.org
20. Martinez, V., et al., Viral vectors for the treatment of alcoholism: Use of metabolic flux analysis for cell cultivation and vector production. Metabolic Engineering, 2010. 12(2): p. 129-137.
21. WO2009007641—CULTURE MEDIUM FOR HAEMOPHILUS INFLUENZAE TYPE B
22. WO2008134220—CULTURE MEDIA FOR DEVELOPMENTAL CELLS CONTAINING ELEVATED CONCENTRATIONS OF LIPOIC ACID
23. WO2009CA01342 20090922—CULTURE MEDIUM FOR MYOBLASTS, PRECURSORS THEREOF AND DERIVATIVES THEREOF
24. WO2008035110—Stem Cell Culture Medium and Method
25. WO2004101808—GENOMIC AND PROTEOMIC APPROACHES FOR THE DEVELOPMENT OF CELL CULTURE MEDIUM
26. US2008248515—Optimizing culture medium for CD34<+>hematopoietic cell expansion
27. US2009061516—ANIMAL CELL CULTURE MEDIA COMPRISING NON ANIMAL OR PLANT DERIVED NUTRIENTS
28. MX2009004974—RATIONALLY DESIGNED MEDIA FOR CELL CULTURE
29. WO2008009642—CELL CULTURE MEDIA
30. WO2009149719—THE USE OF EXTRACT OF SELENIUM ENRICHED YEAST (Se-YE) IN MAMMALIAN CELL CULTURE MEDIA FORMULATIONS
31. Chung, B. K. S., et al., Genome-scale metabolic reconstruction and in silico analysis of methylotrophic yeast Pichia pastoris for strain improvement. Microbial Cell Factories, 2010. 9.
32. Celik, E., P. Calik, and S. G. Oliver, Metabolic Flux Analysis for Recombinant Protein Production by Pichia pastoris Using Dual Carbon Sources: Effects of Methanol Feeding Rate. Biotechnology and Bioengineering, 2010. 105(2): p. 317-329.
33. von Kamp, A. and S. Schuster, Metatool 5.0: fast and flexible elementary modes analysis. Bioinformatics, 2006. 22(15): p. 1930-1931.
34. Pichia fermentation process guidelines, Invitrogen life sciences, www.invitogen.com/pichia
35. Schuster, S, and H. Claus, On Elementary Flux Modes in Biochemical Reaction Systems at Steady State. Journal of Biological Systems, 1994. 2(2): p. 165-182.
36. Schilling, C. H. and B. O. Palsson, Assessment of the metabolic capabilities of Haemophilus influenzae Rd through a genome-scale pathway analysis. Journal of Theoretical Biology, 2000. 203(3): p. 249-283.
37. Shlomi, T., et al., A genome-scale computational study of the interplay between transcriptional regulation and metabolism. Molecular Systems Biology, 2007. 3.
38. Stelling, J., et al., Metabolic network structure determines key aspects of functionality and regulation. Nature, 2002. 420(6912): p. 190-193.
39. Trinh, C. T., et al., Design, construction and performance of the most efficient biomass producing E-coli bacterium. Metabolic Engineering, 2006. 8(6): p. 628-638.
40. Forster, J., A. K. Gombert, and J. Nielsen, A functional genomics approach using metabolomics and in silico pathway analysis. Biotechnology and Bioengineering, 2002. 79(7): p. 703-712.
41. van Os, J., Rutten, B. P. F. & Poulton, R. Gene-Environment Interactions in Schizophrenia: Review of Epidemiological Findings and Future Directions. Schizophrenia Bulletin 34, 1066-1082 (2008)

The following claims present additionally a preferred embodiment of the present invention.

1. The process for determining optimal cell culture medium composition characterized by comprising the following steps:
a) select the target biological structure;
b) establish the set of K elementary cellular functions, which set autonomous and modular biologic functions, which combined together results into the function of the target biological structure;
c) establish the set of N medium factors, which determine the environment of the target biological structure;
d) build a Functional Enviromics Map, which represents the intensity of activation or repression of each of the K elementary cellular functions by each of the N medium factors using experimental data of the exometabolome of the supernatant of fresh and/or spent culture medium samples;
e) optimize the composition of cell culture media oriented to activate or repress one or multiple elementary cellular functions using functional enviromics maps.

2. The process according to claim 1, wherein the construction of the functional enviromics map in step d) comprises the following steps:
execute a first run of cultivation experiments comprising a number of cultivations equal or higher than the number of medium factors plus one (N+1), wherein each cultivation is performed in a different culture medium composition, wherein combinations of low and high levels of medium factors are screened;
acquire initial and end-point biomass, product and exometabolome data or partial exometabolome data for each cultivation experiment performed;
determine a subset of active elementary cellular functions, whose relative weighting factors, λⱼ, are higher than zero by regression analysis of exometabolome data or derived exometabolome data against medium composition data using the following linear model,
$$\begin{matrix}v = \cdot {\sum }_{i = 1}^{K} {λ}_{i} \times {e}_{i} & ( 1 ) \\ {λ}_{i} = \cdot {\sum }_{j = 1}^{N} {I}_{j , i} \times {FAC}_{j} & ( 4 )\end{matrix}$$
with v a vector whose elements represent the rate of change of measured exometabolome components, I_{i,j} are the intensity parameters of activation of elementary cellular function j by the medium factor i determined by regression analysis;
execute a second run of cultivation experiments comprising a number of cultivations equal or higher than the number of active cellular functions plus one, wherein each cultivation is performed in a different culture medium composition, wherein each medium composition is set in order to screen low and high values of weighting factors of active elementary cellular functions using the intensity parameter values, I_{i,j} previously determined, such as to identify the subset of active cellular functions that are controlled by medium factors;
build a functional enviromics map from the data collected in all previous steps by linear regression analysis using the formulas (3a) and (3b) and organize the data in the form of a functional Enviromics map, wherein intensity values I_{i,j} determined with the first run of experiments are refined with the data of the second run of experiments and then put into the form of a N×K data array:
Functional Enviromics map={I_{i,j}};

3. The process according to claim 1 wherein the optimization of culture media composition in step e) comprises the following steps:
formulate elementary cellular function specific medium compositions using the functional enviromics data matrix, wherein variations in medium factor values Δ(FACⱼ) result in variations of the relative weight of elementary cellular functions Δ(λᵢ) according to the formula
Δ(λᵢ)=·I_{j,i}×Δ(FACⱼ) (5)
formulate culture medium compositions to enhance or repress a single elementary cellular function j using formula (4) applied to the jth column of the functional enviromics data array;
formulate culture medium compositions to engineer cellular metabolism by enhancing or repressing critical sets of elementary cellular function using formula (4) applied to multiple column of the functional enviromics data array simultaneously.

4. The process according to any of previous claims, wherein the target biological structure is a cell tissue, a whole cell, an organelle, or a coherent set of biochemical transformations that represent a given cellular function.

5. The process according to any of the claims 1-3, wherein the target biological structure is genetically modified, including genetic modifications oriented to the activation or repression of elementary cellular functions.

6. The process according to any of the claims 1-3, wherein medium factors are physicochemical properties of solid and/or liquid and/or gaseous mixtures of essential nutrients and/or micronutrients and/or biologically functional molecules, or rate of release of said compounds or feeding rate of said compounds.

7. The process according to any of the claims 1-3 and 6, wherein the physicochemical properties comprise temperature and/or pressure and/or pH and/or ionic strength and/or concentration and/or activity and/or osmolality and/or osmolarity and/or related properties.

8. The process according to any of the claims 1-3 and 6, wherein essential nutrients and/or micronutrients and/or functional molecules comprise inorganic and/or organic materials, including salts and/or vitamins and/or metabolic co-factors and/or antibiotics and/or carbohydrate material and/or lipid material and/or proteinaceous material and/or nucleotide material and/or signalling proteins and/or inhibiting molecules of enzyme activity and/or activating molecules of proteins and/or gene transcription modulators and/or interferent ribonucleic acid and/or complex mixtures of said materials with known or unknown composition including sera or hydrosylates of pure or complex organic materials.

9. The process according to any of previous claims wherein the culture medium formulation is determined by the values of N medium factors using the following formula:
Culture medium formulation={FACⱼ}, j=1, . . . ,N,
with FAC the value of medium factor j.

10. The process according to any one of previous claims wherein the target biological structure is determined by q biochemical reactions and K elementary cellular functions using the following formula:
Target biological structure={eᵢ}, i=1, . . . ,K.
with eᵢ a vector of q elements whose values represent the weighting factor of each biochemical reaction in the elementary cellular function i.

11. The process according to any one of previous claims wherein the elementary cellular functions are obtained from a biochemical network of said target biological structure, wherein the biochemical network is sub-divided into K functional sub-networks comprising a subset of biochemical transformations, wherein such sub-networks are obtained manually and/or automatically.

12. The process according to any of the previous claims, wherein elementary cellular functions are obtained from genome scale reconstruction of the biochemical network of said target biological structure, wherein the working set of K elementary cellular functions may be pre-reduced using transcriptome data and/or proteome data and/or endometabolome data and/or thermodynamic data in case these data are available.

13. The process according to any of the previous claims, wherein the functional enviromics map is determined by serial and/or parallel culture experiments performed in shake-flasks, T-flasks, reactors, microplates, microbioreators or phenotypic microarrays.

14. The process according to any of the previous claims, wherein the functional enviromics map is determined by exometabolome assays, comprising, analysis of the supernatant of fresh or spent culture medium samples by chromatography techniques, such as liquid chromatography (LC) or gas chromatography (GC), NMR techniques, such as 1H-NMR or 13C-NMR, mass spectrometry techniques (MS) or chromatography coupled to mass spectrometry, such as GC-MS or LC-MS, or by combinations of said measurement techniques.

15. The process according to any of the previous claims, wherein a reduced set of active elementary cellular functions are identified by linear or nonlinear regression analysis, wherein variance or co-variance of exometabolome data or derived exometabolome data is maximized, wherein correlation between exometabolome data or derived exometabolome data and medium factors values is maximised, wherein elementary cellular functions are ranked according to their correlation or sensitivity to medium factors values.

16. The process according to any of the previous claims, wherein the functional enviromics map is determined by a high-throughput automated system, wherein cultivation devices, analytical exometabolome devices and computational algorithms are interfaced in a physical device to produce high-throughput functional enviromics maps.

17. The process according to any of the previous claims wherein the target elementary cellular function i associated to product quantity and/or product quality increases its relative weight Δ(λᵢ) between 60 to 100%.

18. Chemically defined culture media formulations for the cultivation of the yeast Pichia pastoris characterized by enhancing the heterologous protein expression function between 60 and 100%, obtained through the process described in claims 1-17, comprising:
a) an aqueous solution of trace elements composed by CuSO₄.5H₂O, 12.0 g/L, NaI, 0.16 g/L, MnSO₄.H₂O, 6.00 g/L, Na₂MoO₄.2H₂O, 0.40 g/L, H₃BO₃, 0.001 g/L, CoCl₂.6H₂O, 0.25 g/L, ZnCl₂, 40.0 g/L, FeSO₄.7H₂O, 3.25 g/L, Biotine, 0.4 g/L, H₂SO₄, 10.0 mL/L; and
b) mixtures of solution a) with a complementary basal aqueous solution composed by H₃PO₄ 85%, 26.70 ml/L, CaSO₄.2H₂O 0.93 g/L, K₂SO₄ 18.20 g/L, MgSO₄.7H₂O 14.90 g/L, KOH 4.13 g/L, or other complementary basal aqueous solutions.

19. Use of the process described in claims 1-17 to increase the quantity and/or quality of tissues and/or cells and/or viruses and/or cellular components and/or proteinaceous material and/or carbohidrate material and/or nucleotide material and/or lipid material and/or primary metabolites and/or secondary metabolites or mixtures of said products in biological production processes, such as in biofuels production, or in vaccines production, or in drugs production, or in biopolymers production or in the production of precursors of said products.

20. Use of the process described in claims 1-17 for optimization of the composition of cell culture media of Plantae or Animali cell lines or other eukaryotic unicellular or multicellular organism such as Yeasts and Fungi.

21. Use of the process described in claims 1-17 for optimization of the composition of cell culture media of prokaryotic organisms.

22. Use of the process described in claims 1-17 for optimization of the composition of cell culture media of stem cells.

23. Use of the process described in claims 1-17 for the identification of biomarkers of cellular functions.

24. Use of the process described in claims 1-17 for the design of drugs or for the optimization of drug mixtures oriented to modify cellular functions associated to illness conditions.

25. Biomarkers identifiers comprising the process described in claims 1-17.

26. Drug design system comprising the process described in claims 1-17.
